(19) United States
(12) Patent Application Publication
(10) Pub. No.: US 2012/0277788 A1
(43) Pub. Date: Nov. 1, 2012

(54) MEDICAL DEVICE FOR INSERTION INTO A HOLLOW ORGAN AND METHOD FOR PRODUCING SUCH A DEVICE

(75) Inventor: Giorgio Cattaneo, Karlsruhe (DE)

(73) Assignee: ACANDIS GMBH & CO. KG, Pfinztal (DE)

(21) Appl. No.: 13/512,847

(22) PCT Filed: Dec. 1, 2010
(86) PCT No.: PCT/EP2010/007302

§ 371 Date: Jun. 26, 2012

(30) Foreign Application Priority Data
Dec. 1, 2009 (DE) 10 2009 056 450.0

(51) Int. Cl. A61F 2/01

(52) U.S. Cl. 606/200

(57) ABSTRACT

A medical device for insertion into a hollow organ, the device having a hollow body that has a braid of wire elements having a series of terminal meshes which delimit an axial braid end, wherein the terminal meshes have outer wire elements forming a terminating edge of the braid and transition into inner wire elements arranged within the braid. The device is characterised in that a first section of the terminating edge and a second section of the terminating edge each have several outer wire elements which form together a peripheral edge of the terminating edge, which is adjusted such that the axial braid end of the hollow body can be refracted into a supply system. A method for producing such a device is also disclosed.

The invention relates to a medical device for insertion into a hollow organ and to a method for producing such a device. A device of the type in question, with the features of the preamble of claim 1, is known, for example, from DE 101 27 602 A1.

For the treatment of aneurysms and stenoses and for the expansion of clots, lattice structures are suitable that have very fine meshes. By means of the fine meshes, the flow conditions in the aneurysm can be influenced, in particular by slowing down the flow through the aneurysm with the aim of effecting the coagulation and sclerosis of the aneurysm. The finely meshed lattice structure also promotes the rapid growth of cells (endothelialization). Moreover, in the expansion of stenoses and of clots, particles are efficiently blocked by the fine meshes.

A very fine mesh structure is achieved in particular by braids, which are therefore especially suitable for this type of use.

Braids are also used in the endovascular area whenever increased flexibility is demanded. For example, baskets for protecting against distal embolism or for removing clots can be produced in the form of braids. The good flexibility of braids also permits treatment in highly tortuous areas, for example in the brain. Baskets can also be used in other areas, for example in the bladder in order to remove stones. The flexibility is also advantageous in connection with the treatment of aneurysms or stenoses when these are located at highly tortuous vascular sites.

It is known to produce braids with an open structure. This means that the wires of the braid have free ends. In this way, the wires can be braided as a long strand, which is then cut to the suitable length. In this way, different units are formed with in each case open or free wire ends. This production technique is suitable if the braid has many wires and if individual production of individual units is uneconomical. Braids with a large number of wires are very finely meshed and have the aforementioned advantages.

Alternatively, braids can be produced with closed loops. This requires individual production of each braid. The braids thus produced have the advantage that the wire ends rounded off by the loops are atraumatic and reduce the risk of injury to the vessel wall.

An example of a braid with closed loops is disclosed in the aforementioned DE 101 27 602 A1. Said document describes a stent for implantation in the human body, with a hollow cylindrical body which is produced from a braid. At the braid ends, the free ends of the wires of the braid are brought together and connected in such a way that loops or meshes are formed at the braid ends. The loops at the braid ends lead to a terminating edge of the braid with a jagged contour.

Braids in which the wires end freely, and also braids with closed loops at the braid ends, as are described in DE 101 27 602 A1 for example, have the disadvantage that the free wire ends or the loops impede or even completely prevent a retraction of the braid into a catheter after the braid has been released from the catheter into the vessel. In braids with free wire ends at the braid end, there is the further problem, as regards the retractability of the braid, that a guide wire cannot in practice be connected to the braid for retraction thereof. Even if the braid were to be connected at some point to a guide wire that retracts the braid into the catheter, the free wires would catch on the catheter opening. This would damage the braid and the catheter. It is therefore not possible to draw the braid back.

It is also not possible for a braid ending with closed loops to be retracted into the catheter after it has been fully released. Such a braid has several tips at the braid end, and the tips become jammed during the retraction into the catheter. This is especially the case if the braid is connected to a guide wire at a single location.

The object of the invention is to make available a medical device for insertion into a hollow organ, which device comprises a braid, of which the axial braid end is designed such that the medical device can be drawn back into a delivery system, for example into a catheter, even when the medical device has been released completely or almost completely from the delivery system. The invention also has the object of making available a method for producing such a device.

According to the invention, the object is achieved, in respect of the device, by the subject matter of claim 1, and, in respect of the method, by the subject matter of claim 22.

The invention is based on the concept that a medical device for insertion into a hollow organ comprises a hollow body that has a braid of wire elements with a series of terminal meshes, which delimit an axial braid end. The terminal meshes comprise outer wire elements, which form a terminating edge of the braid and merge into inner wire elements arranged within the braid.

A first section of the terminating edge and a second section of the terminating edge each have several outer wire elements which together form a peripheral margin of the terminating edge. The margin is adapted in such a way that the axial braid end of the hollow body can be drawn into a delivery system.

The outer wire elements of the first section for forming the terminating edge are arranged directly one after another along the latter. The outer wire elements each have a first axial component, which extends in the longitudinal direction L of the hollow body.

The outer wire elements of the second section for forming the terminating edge are arranged directly one after another along the latter. The outer wire elements each have a second axial component, which extends in the longitudinal direction L of the hollow body. The second axial component is counter to the first axial component, wherein the two axial components are in relation to the same rotation direction of the margin.

The hollow body generally has an elongate shape with a longitudinal direction and is suitable to be inserted through a delivery system, for example through a catheter, into a hollow organ, in particular into a human hollow organ. The longitudinal direction of the hollow body corresponds to the direction in which the hollow body is moved through the delivery system and released in the hollow organ. The expression longitudinal direction of the hollow body thus corresponds to the expression direction of movement of the hollow body in the delivery system. The direction of movement of the hollow body in the delivery system can comprise the advance movement during the release of the hollow body and also the rearward movement during the retraction of the hollow body into the delivery system.

The expression terminal meshes is understood as meaning the outer meshes of the braid that delimit a braid end. In an elongate hollow body, the braid end is an axial braid end. The outer wire elements of the terminal meshes are the wire elements which are arranged on the outside of the terminal meshes and which represent the limit of the braid end. The outer wire elements form a terminating edge of the braid and merge into inner wire elements, which are arranged within the braid.

The outer wire elements can thus also be designated as edge-forming wire elements and the inner wire elements as braid-forming wire elements.

The first section of the terminating edge and the second section of the terminating edge each form a part-edge, which delimits the braid in the longitudinal direction of the hollow body. Each section has several outer wire elements or is formed by several outer wire elements which together form a peripheral margin of the terminating edge. The peripheral margin is adapted in such a way that the axial braid end of the hollow body can be drawn into a delivery system. This means that the hollow organ can be retracted in the longitudinal direction with the peripheral margin into the delivery system after the hollow body has been released completely or at least substantially from the delivery system.

The outer wire elements of the two sections for forming the terminating edge are in each case arranged directly one after another along the latter. This means that the outer wire elements of the first section form a first unit of the peripheral margin, which first unit is distinguished by the fact that the outer wire elements of this first unit each have a first axial component, which extends in the longitudinal direction of the hollow body.

The same applies analogously to the outer wire elements of the second section which, for forming the terminating edge, are arranged directly one after another along the latter and form a second unit of the peripheral margin. The outer wire elements of the second section are distinguished by the fact that they each have a second axial component, which extends in the longitudinal direction of the hollow body. The two axial components are arranged in opposite directions, wherein the two axial components are in relation to the same rotation direction of the margin. This means that one axial component faces forward in the longitudinal direction of the hollow body and the other axial component faces rearward in the longitudinal direction of the hollow body. In other words, one of the two axial components faces in the proximal direction, i.e. in the direction of the user, and the other of the two axial components faces in the distal direction, i.e. away from the user or physician. The different direction of the axial components is expressed by the fact that they are in relation to the same rotation direction of the margin. The same rotation direction of the margin is understood, for example, as the movement of an imaginary point on the margin in one and the same direction. It does not matter whether the movement of this imaginary point takes place clockwise or counter clockwise on the margin. For the definition of the direction of the axial components, it suffices if the rotation direction or the imaginary rotation is in the same sense.

By means of the novel type of braiding, the braid is structured such that the end contour of the braid, i.e. the contour in the area of the terminating edge, has, at least at one axial braid end, no protruding edges, or no edges protruding counter to the direction of retraction of the braid end into the delivery system. Rather, by means of the peripheral margin of the terminating edge and by means of the outer wire elements oriented according to the invention, the braid end can be retracted into the delivery system without parts of the braid end protruding radially outward beyond the opening of the delivery system and impeding the retraction. The outer wire elements form a common compact terminating edge.

The function of the retractability into a delivery system, particularly into a catheter, after the medical device has been completely released can be used both in implants and also in medical articles that are released only temporarily in a vessel, for example baskets, in particular clot catchers. In the case of implants, the advantage is that the positioning of the system, for example the positioning of stents, can be corrected after the release thereof. The implant can be deployed completely in the vessel and bear on the vessel wall. After checking the position of the implant, the user can draw the system back in again if incorrect positioning is observed and can then release the system anew, for example in front of an aneurysm or a stenosis. In the case of temporarily released devices, for example baskets or filters, the retractability function serves to ensure that the particles, for example clots, caught in the basket or filter can be removed from the vessel or from other hollow organs.

The invention also has the advantage that the braid can, at a single location of the braid end, be connected to an actuation element for retraction, for example to a guide wire, in order to ensure the retractability function. By virtue of the invention, it is not necessary (although the possibility is not ruled out) for the individual meshes to be connected to the guide wire in order thereby to achieve a radially inward orientation of the terminal meshes, so as to support the retractability function. In order to orient the terminal meshes inward, connecting wires would be needed between the terminal meshes and the guide wire, and this would result in the proximal end of the basket being obstructed by the connecting wires. The connecting wires would, for example, impede the catching of a clot. By contrast, the invention ensures that a braid configuration with an open braid lumen, particularly in the case of implants such as stents, or with an open proximal braid end, for example in baskets, whose distal end is closed, can be retracted into the delivery system after being completely released. The invention can also be applied to braid configurations with a closed braid lumen.

As regards the method according to the invention, the invention is directed to making available a method for producing a medical device, in which method a hollow body is braided from wire elements, and a first series of terminal meshes arranged next to one another and delimiting an axial braid end is formed. Each terminal mesh comprises at least one outer wire element. During the braiding, the outer wire elements of the terminal meshes are arranged such that they together form a terminating edge of the braid end, wherein inner wire elements arranged in the braid are deflected in order to form the outer wire elements. The deflection is such that the direction of winding and/or the direction of the axial component with respect to the longitudinal axis of the inner wire elements is changed. The change in the direction of winding or the change in the direction of the axial component with respect to the longitudinal axis L takes place at the transition from the inner to the outer wire elements.

In a preferred embodiment of the invention, the outer wire elements of the first section have a first circumferential component, and the outer wire elements of the second section have a second circumferential component, wherein the first and second circumferential components extend in the same circumferential direction of the hollow body. The circumferential direction of the hollow body is generally the direction in which the wall of the hollow body extends. For example, in a cylindrical hollow body, the circumferential direction is the direction in which the jacket surface of the hollow body extends. In contrast to the circumferential direction, which is generally in relation to the wall of the hollow body, the rotation direction of the margin is understood as the extent resulting from the contour of the margin. For example, the rotation direction of the margin corresponds to the circumferential direction of a hollow cylindrical body if the margin extends in a plane that is perpendicular to the longitudinal axis of the hollow body. If, for example, the plane in which the margin lies is inclined with respect to the longitudinal axis, the rotation direction of the margin and the circumferential direction of the hollow body are different.

In another preferred embodiment of the invention, the terminating edge is arranged obliquely with respect to the longitudinal direction of the hollow body. This further improves the retraction of the hollow body or of the medical device into a delivery system, since the oblique terminating edge results in the formation, in the longitudinal direction, of a proximal tip of the braid end, which proximal tip is moved first into the delivery system during the retraction and performs a guide function.

In a particularly preferred embodiment of the invention, at least one first outer wire element of a terminal mesh extends into the area of another terminal mesh, wherein the first outer wire element and at least one second outer wire element of the other terminal mesh overlap each other and are arranged together along the terminating edge. The overlapping of the two wire elements means that, in the area of the other terminal mesh, they together form the terminating edge and are arranged together along the latter. This arrangement of the outer wire elements is a preferred possibility for the design of a retractable peripheral margin or of such a terminating edge. The overlapping of the wire elements takes place mainly in the first section or in the second section of the terminating edge.

Preferably, the at least one first outer wire element and the at least one second outer wire element are connected. In the case of the wire elements arranged together along the terminating edge and overlapping each other, the connection of the wire elements has the advantage that the braid end, in particular the terminating edge, is stable. This further improves the retractability function. Moreover, a particularly smooth peripheral margin is formed which, in addition to being easy to retract, is also atraumatic.

In another preferred embodiment of the invention, the terminal meshes are formed by loops, which are staggered along the terminating edge and overlap each other. To form the in particular interconnected outer wire elements at the terminating edge, the loops are brought together at different locations that are arranged in succession along the terminating edge. A configuration of the braid end is thus achieved in which several wire loops are nested one inside another, and specifically such that the loops merge into one another at different locations along the terminating edge. A staggered arrangement of loops is achieved in this way, the staggered arrangement extending along the terminating edge.

The configuration of the terminal meshes by means of the above-described loops affords a simple possibility by which the braid end is formed and a smooth peripheral margin is obtained. In addition, the loop configuration provides a wide range of variation, since the staggered arrangement of the loops can be modified in various ways.

In another preferred embodiment, the inner wire elements are guided to the terminating edge. Alternatively, the inner wire elements are branched off from the terminating edge. This provides two possible ways of strengthening the terminating edge when forming a peripheral retractable margin. In the first possibility, starting from a thin section of the terminating edge, for example a section with only a single outer wire element, the inner wire elements are delivered to the terminating edge and the latter is thereby successively strengthened, such that the terminating edge with each subsequent terminal mesh has more outer wire elements and thus becomes thicker. In the second possibility, starting from a strengthened terminating edge or from a strengthened section of the terminating edge, the latter is reduced in strength by means of wire elements being branched off from the terminating edge and being guided onward as so-called inner wire elements in the braid. In both alternatives, the outer wire elements of the terminating edge merge into the branched-off or delivered inner wire elements.

The merging outer and inner wire elements of one and the same terminal mesh are sections of a continuous wire and can also generally be designated as inner or outer wire elements sections or as wire sections.

At the transition from the inner wire elements to the outer wire elements or, conversely, at the transition from the outer wire elements to the inner wire elements, the direction of winding and/or the direction of the axial component with respect to the longitudinal axis of the hollow body is changed. In terms of the braiding technique, the change in direction of the wire elements makes it easier to bring the individual wires together in a common terminating edge.

The inner wire elements and the outer wire elements can have the same braiding angle, as a result of which the terminating edge and the wires located in the braid do not collide. In addition, the same braiding angle avoids the structure distorting when retracted into the delivery system. This does not exclude the possibility of the braiding angle changing in the braid. It suffices if the inner wire elements adjoining the terminating edge, or the inner wire elements near the edge, and the outer wire elements have the same braiding angle.

The retractability into the delivery system is improved and facilitated particularly if all the wires or wire elements located at the same axial level have the same braiding angle. This applies to the inner wire elements and to the outer wire elements. The braiding angle is the angle between the wire element and a projection of the longitudinal center axis of the device. The angle can be between 0°, in particular more than 0°, and 90°. Depending on the spiral direction of different wire elements or wire sections, the braiding angle at the same absolute value can be differently oriented, for example in the area of the deflection of a wire element.

Preferably, all the inner wires and outer wires in the entire braid area of the terminating edge have the same braiding angle. The braid area of the terminating edge is the area of the medical device extending between two planes, of which one extends perpendicularly with respect to the axis of the braid and the other extends through the tip and through the apex of the terminating edge. This is therefore the entire area, limited in the longitudinal direction, to which the terminating edge belongs. It is preferable if all the wires in the braid area of the terminating edge, or at the same level in the braid area of the terminating edge, have a similar angle (absolute value). The difference between the braiding angle of two wires is preferably at most 20° 15° 10° 8° 6° 5° 4° 3° 2° 1°.

At the deflection locations, where the inner wire elements merge into the terminating edge, the wires have a changing braiding angle. The deflection location can be a kink. Preferably, the deflection location has a radius of less than 1 mm, 0.8 mm, 0.6 mm, 0.4 mm, 0.3 mm, 0.2 mm, 0.1 mm. In this way, the wire can change the spiral direction on a short path. The wires, preferably all wires which are deflected upstream and downstream of the deflection location, i.e. upstream and downstream of the radius of the kink, preferably have a braiding angle difference (absolute value) of at most 20° 15° 10° 8° 6° 5° 4° 2° 1°.

The interconnected outer wire elements of the terminal meshes can be connected by a form fit, in particular by twisting and/or intertwining, and/or by an integral bond, in particular by adhesive bonding, welding or soldering, and/or by mechanical connecting means, in particular by coils and/or sleeves and/or wires. In the connecting technique, sleeves are of advantage, particularly sleeves that are visible by X-ray, preferably platinum/iridium sleeves. In a preferred variant, the sleeves are mechanically crimped. However, the sleeves can also be welded. The placing of X-ray-visible material on the terminating edge, optionally on the entire edge, has the advantage that the opened braid end and its position can be easily seen.

In a preferred embodiment of the invention, the number of the outer wire elements of the first section and the number of the outer wire elements of the second section in each case increases toward a front area of the terminating edge in the longitudinal direction of the hollow body. The number of the outer wire elements of the first section and the number of the outer wire elements of the second section are each at a maximum in the front area. For example, in the case of a terminating edge arranged obliquely with respect to the longitudinal direction of the hollow body, the front area of the terminating edge is the area of the front tip or the proximal area of the terminating edge. Starting from a terminal mesh arranged remote from the front area, the number of the outer wire elements increases with each additional terminal mesh, and specifically in the direction of the front area. There, the number of the outer wire elements is at its maximum. This applies to each of the two sections. This embodiment provides a structure that is stable in the front area of the terminating edge and that improves the guide function of the terminating edge upon retraction into the delivery system.

The outer wire elements of the terminal meshes can be brought together in a front terminal mesh. This also increases the stability of the terminating edge in the front area: The outer wire elements can form a single group, which is deflected in the area of the front terminal mesh. This means that the free wire ends of the respective wires that form the outer wire elements are arranged in the area of the other braid end. There, the free wire ends can be fixed in a conventional manner. It is also possible to arrange the free wire ends as in the document DE 10 2009 006 180.0 going back to the applicant. The content of said application is incorporated in full by reference.

Alternatively, the outer wire elements can form at least two groups, which are connected in the area of the front terminal mesh. This means that the free ends of the associated wires, or those wires that form the respective outer wire elements, are brought together in the area of the front terminal mesh and are connected there. The free wire ends can be connected in a conventional manner.

Preferably, the number of the outer wire elements of the first section and of the second section in each case decreases toward a front area of the terminating edge in the longitudinal direction of the hollow body and is at a minimum in the front area. With regard to the arrangement of the front area, reference is made to the above comments. In contrast to the aforementioned embodiment, the number of the wire elements decreases in the proximal direction of the terminating edge. In other words, starting from the front area of the terminating edge, the number of the outer elements rises with increasing distance until the number of the outer wire elements is at a maximum. The outer wire elements are gathered to form a wire bundle which, in order to strengthen the hollow body, is arranged in the braid and forms a part of the braid. This means that the wire bundle forming the terminating edge is guided into the braid and forms a part of the braid. The resulting strengthening of the braid stabilizes the hollow body.

In another preferred embodiment of the invention, an intermediate area is arranged between the front area and a rear area of the terminating edge. The number of the outer wire elements, starting from the intermediate area, decreases toward the front area and toward the rear area of the terminating edge. The number of the outer wire elements is at a maximum in the intermediate area. The rear area of the terminating edge is that area in which the terminating edge merges into the closed wall of the braid. In the case of an obliquely arranged terminating edge, the rear area of the terminating edge is arranged opposite the front area and spaced apart therefrom, wherein the front area forms a braid tip. This applies to all the illustrative embodiments in which the terminating edge is arranged obliquely. In the aforementioned embodiment, the intermediate area is arranged between the front area and the rear area of the terminating edge and spaced apart from each of these. The associated lateral strengthening of the terminating edge opens up further possible uses of the device.

In another embodiment of the invention, the inner wire elements form inner mesh limits of the terminal meshes of the first section and/or of the second section which, starting from a terminating edge, extend into the braid interior, wherein the terminal meshes are at least in part interconnected at the inner mesh limits. In this embodiment, the connection of the terminal meshes to one another is effected mainly by the inner wire elements and less so by the outer wire elements. It has been shown that, in such a configuration of the invention too, a sufficiently stable terminating edge is achieved which permits retraction of the braid end into a delivery system, without the individual terminal meshes becoming caught on the admission opening of the delivery system.

The outer wire elements of the terminal meshes can be arranged such that they do not overlap. In this embodiment, edges can occur between the individual terminal meshes, but these edges do not impede the retraction of the hollow body into the delivery system, since they extend in the direction of retraction or do not protrude counter to the direction of retraction.

It will be noted that small projections can generally form along the terminating edge. These can be caused, for example, by the twisting of the wires. In the context of the invention, the terminating edge is designated as smooth when there is a substantially continuous profile of the wire elements. This means that no sharp edges arise to impede the retraction of the lattice braid into a delivery system. Individual wire elements can thus protrude in the area of the terminating edge, these wire elements describing a curve which describes such a large radius that the terminating edge is smooth as a whole or continuously smooth. Advantageously, the protruding wire element protrudes at most by an amount that corresponds maximally to the diameter of the wire element. In particular cases, it is also possible that the wire element protrudes beyond adjacent wire elements, the protrusion corresponding at most to three times the wire diameter. It is important that the terminating edge is made continuously smooth so as to permit the retraction into a delivery system.

In another embodiment, the terminal meshes of the first section and/or of the second section in each case have at least two loops, which are arranged next to each other along the terminating edge, wherein at least a third loop overlaps the first loop and the second loop. This embodiment has the advantage that the variously combinable loops permit production of a braid which is designed differently in the area of the terminating edge, such that the mechanical properties, for example the radial force, the flexibility and the mesh fineness, can be adapted very precisely. This applies not only to the area near the outer edge, but also to the entire braid.

In a particularly preferred embodiment of the invention, at least one outer wire element and/or an additional wire element forms a continuation which extends beyond the contour of the terminating edge. The continuation can be used, for example, to connect a guide wire to the hollow body. The continuation can be designed, for example, in the form of a loop, into which a corresponding mating piece of the guide wire is hooked.

The aforementioned features of the various embodiments are design features that can be implemented on the device and that characterize the latter. The aforementioned features of the various embodiments are also disclosed, where appropriate, as method features in connection with the method of production.

The invention is explained in more detail below on the basis of illustrative embodiments and with reference to the attached schematic drawings, in which:

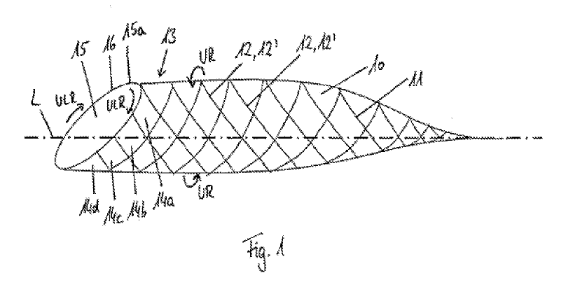

FIG. 1 shows a perspective side view of a medical device in one illustrative embodiment according to the invention, in particular a basket;

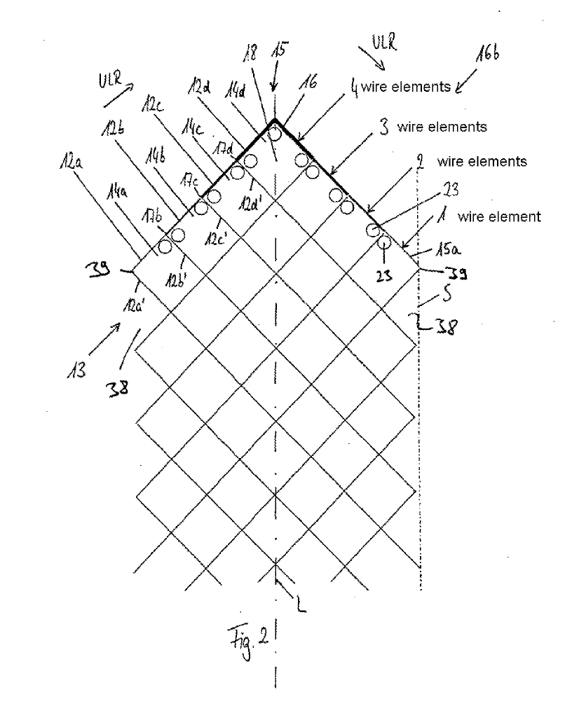

FIG. 2 shows a developed view of the device according to FIG. 1;

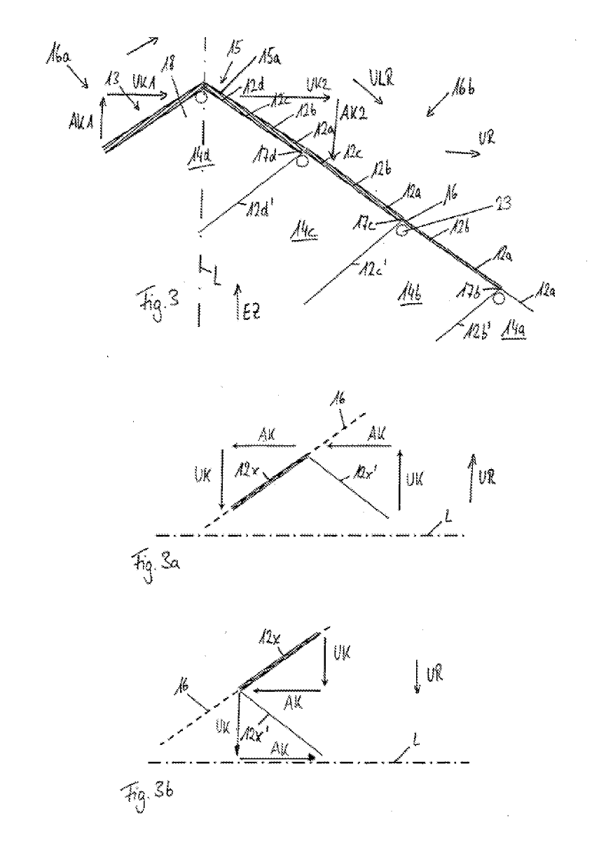

FIG. 3 shows an enlarged detail of the terminating edge of the device according to FIG. 2;

FIG. 3a shows a schematic view illustrating the change in the winding direction of the wire elements of the device according to FIG. 1;

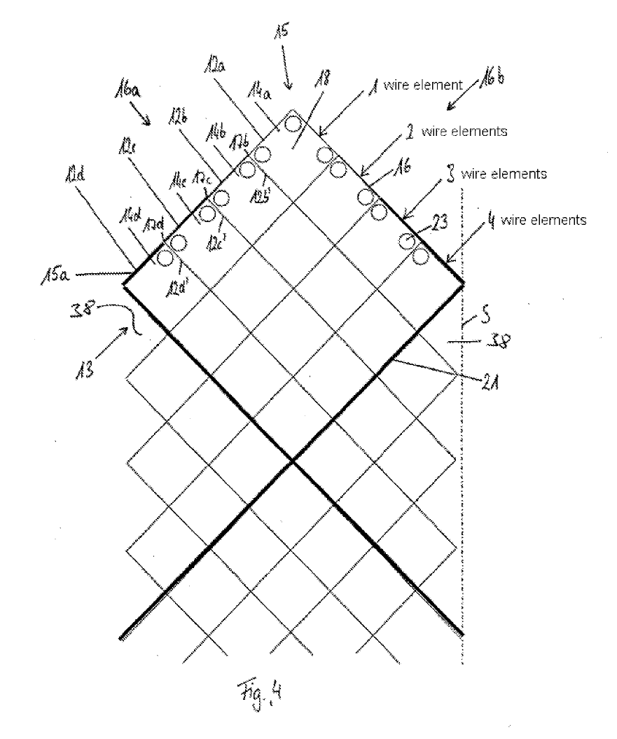

FIG. 3b shows a schematic view illustrating the change of direction of the axial component of the wire elements in the device according to FIG. 4;

FIG. 4 shows the developed view of a medical device in another illustrative embodiment according to the invention;

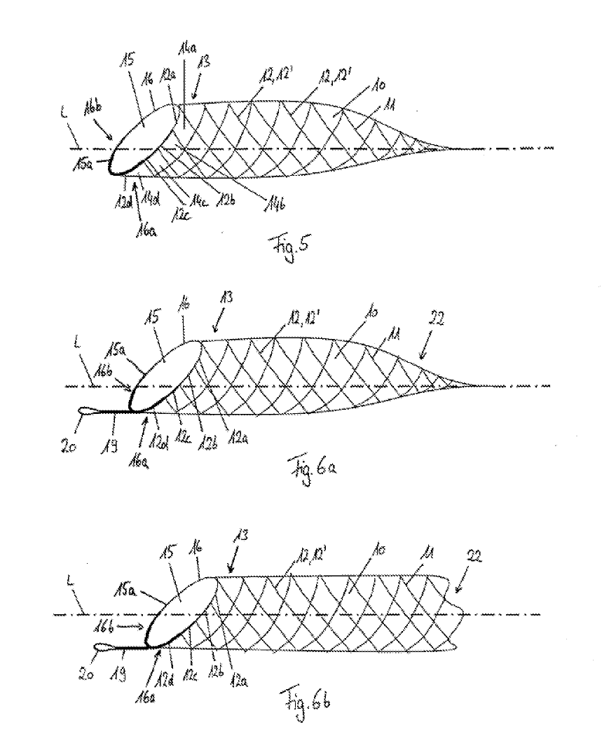

FIG. 5 shows the device according to FIG. 1 with further details in the area of the terminating edge;

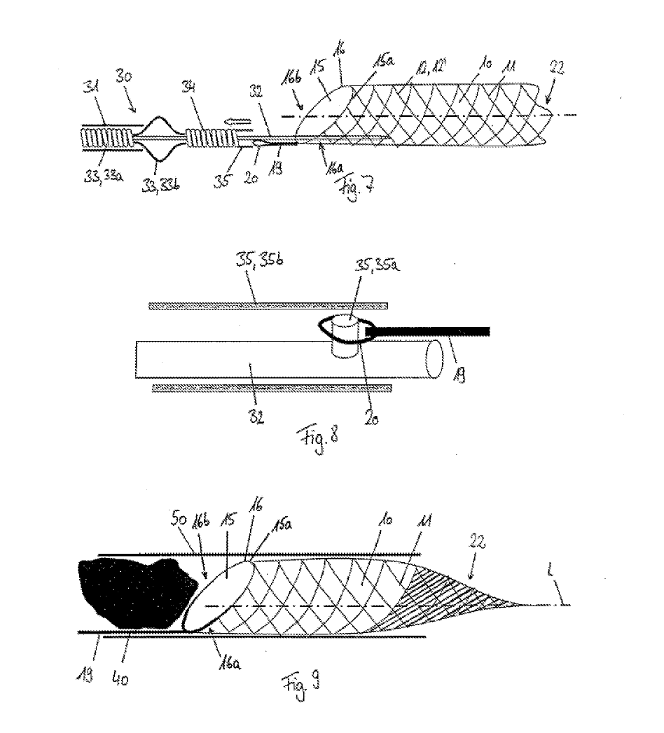

FIG. 6a shows a perspective side view of a device in another illustrative embodiment according to the invention;

FIG. 6b shows a perspective side view of a device in another illustrative embodiment according to the invention;

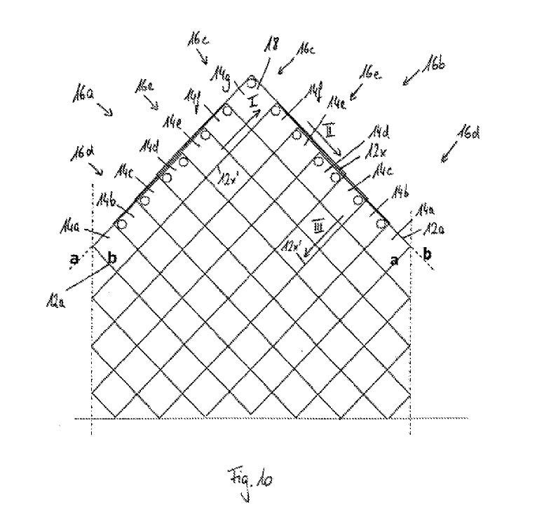

FIG. 7 shows a schematic view of an arrangement comprising the device according to FIG. 6a or FIG. 6b and an associated delivery system;

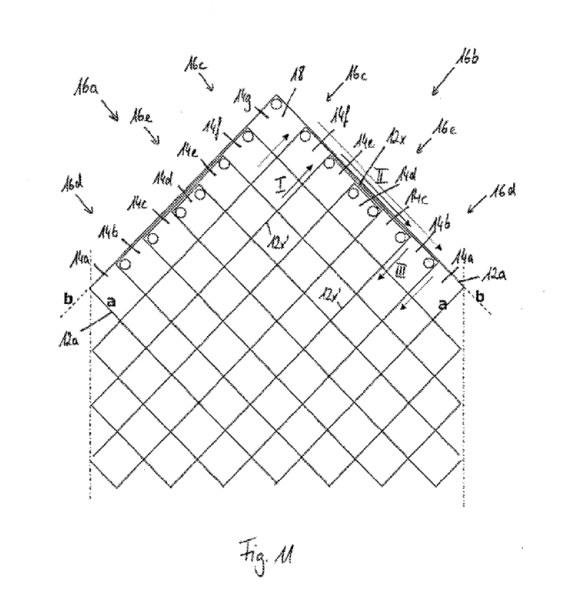

FIG. 8 shows an enlargement of the connecting area between the delivery system and the device according to FIG. 7;

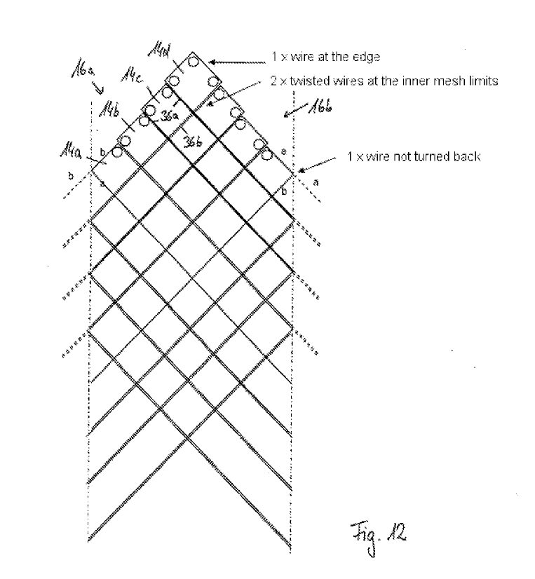

FIG. 9 shows a schematic view of a medical device in use in another illustrative embodiment according to the invention;

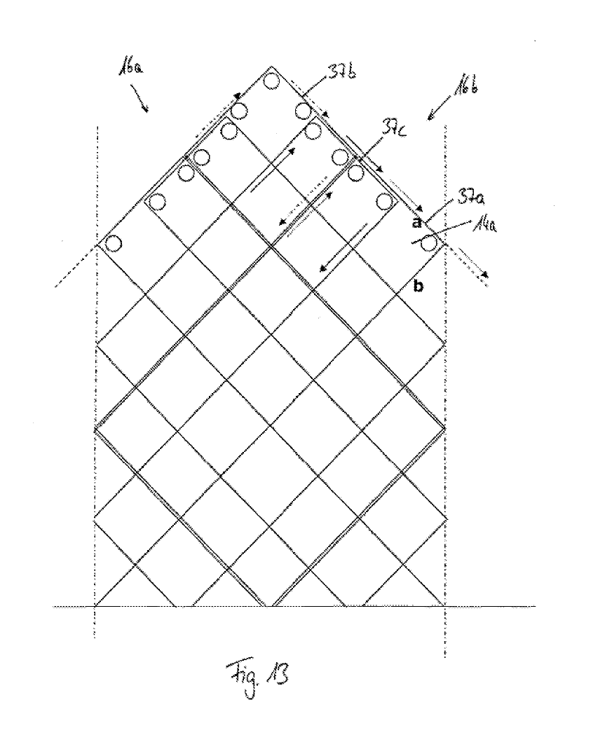

FIG. 10 shows the developed view of a medical device in another illustrative embodiment according to the invention;

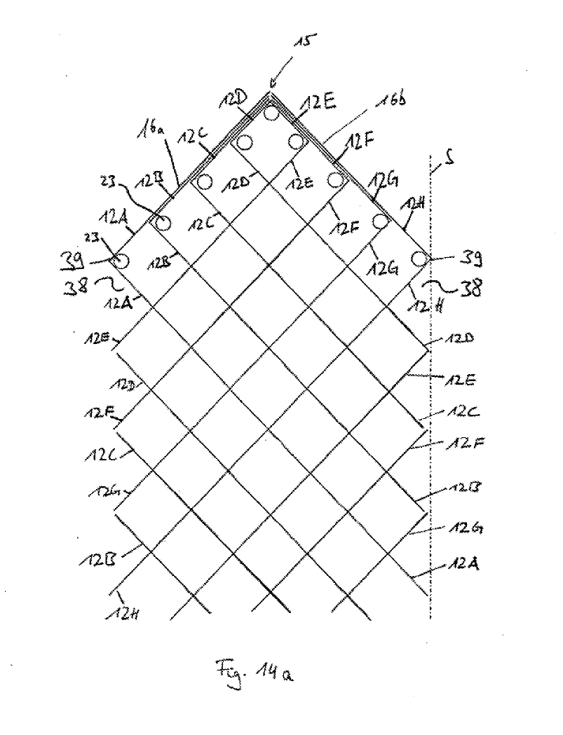

FIG. 11 shows the developed view of a medical device in another illustrative embodiment according to the invention, with an alternative loop configuration;

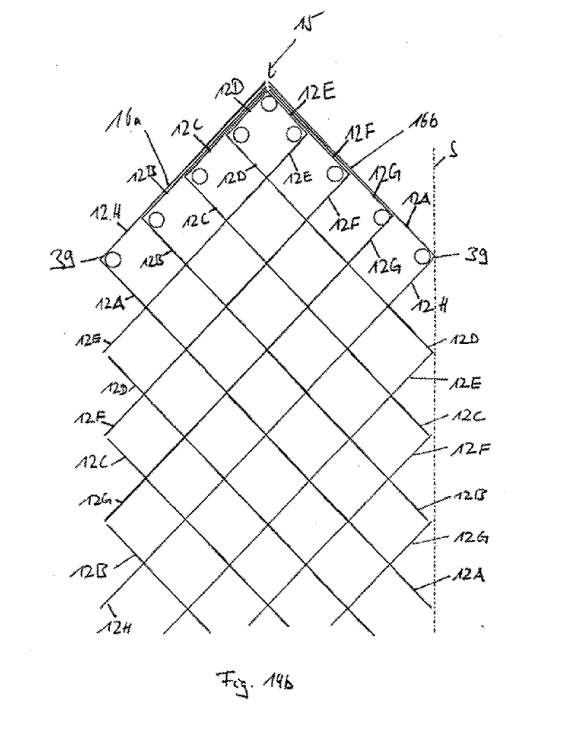

FIG. 12 shows the developed view of a medical device in another illustrative embodiment according to the invention, in which the inner mesh limits are connected;

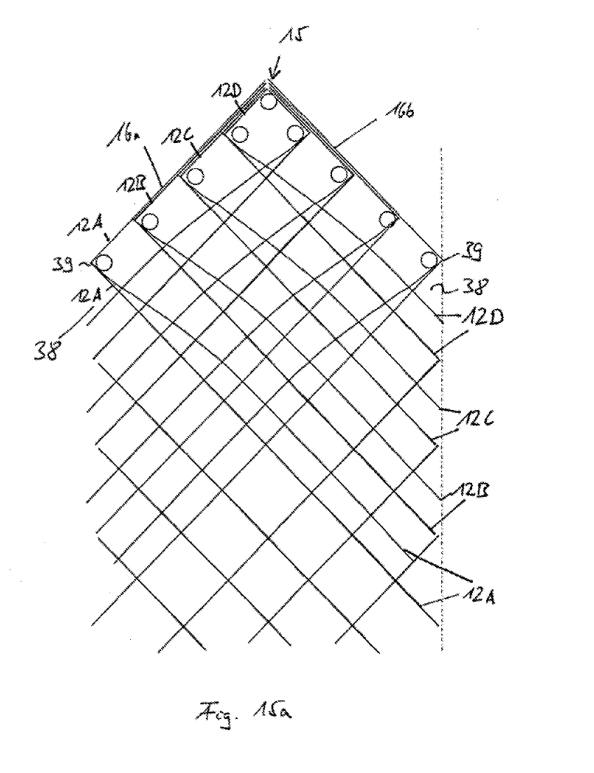

FIG. 13 shows the developed view of a medical device in another illustrative embodiment according to the invention, in which some of the loops are arranged next to each other and some overlap;

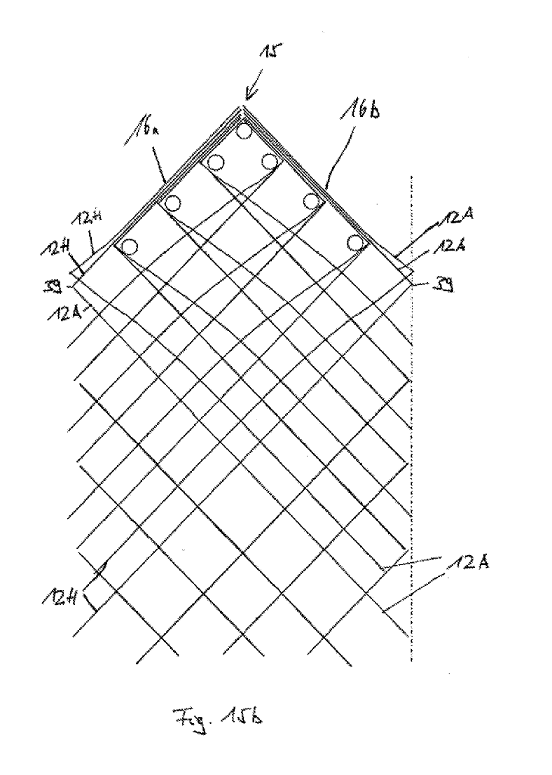

FIG. 14a shows the developed view of a medical device in another illustrative embodiment according to the invention, with deflected first wire elements;

FIG. 14b shows the developed view of a medical device in another illustrative embodiment according to the invention, with non-deflected first wire elements;

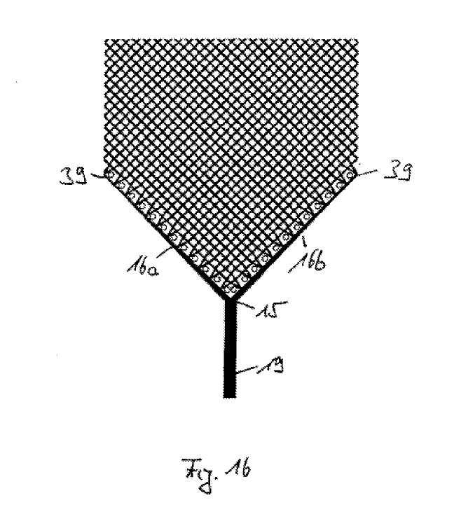

FIG. 15a shows the developed view of a medical device in another illustrative embodiment according to the invention, with multiply wound individual wires and deflected first wire elements;

FIG. 15b shows the developed view of a medical device in another illustrative embodiment according to the invention, with multiply wound individual wires and non-deflected first wire elements; and

FIG. 16 shows the developed view of a medical device in another illustrative embodiment according to the invention, with a multiple wire configuration.

FIGS. 1 to 3 and FIG. 5 show a medical device for insertion into a hollow organ, in particular into a human hollow organ, in an illustrative embodiment according to the invention. The device is a basket which can be used, for example, to remove clots. The invention can also be applied to other medical devices, for example stents, flow dividers, filters and the like. The device is particularly suitable in stent-like systems that are used to influence flow. Systems to influence flow have particularly fine meshes and have small cells. For example, a fine-mesh device has 16, 24, 32, 36, 40, 44, 48, 64, 72, 80, 96 wire elements. In such devices, precise positioning is important. For example, the device is intended to influence or reduce the flow in an aneurysm or at an arteriovenous malformation, while side branches are intended to remain open. It is important for the device to be drawn back again into the catheter if the positioning is not optimal, for example if the flow is influenced inadequately or if side branches are partially occluded. The influence on flow and the occlusion of side branches can be evaluated by angiography. The device can also be a partially covered or completely covered implant. A membrane, preferably a plastic membrane, can partially or completely cover at least one area of the device. If completely covered, this is called a stent graft. Complete coverage can be obtained, for example, using a thin polyurethane film. For this purpose, the braid can be immersed in a polyurethane mixture, for example. The covered implant serves to partially or completely suppress the blood flow, for example in aneurysms or arteriovenous malformations.

The invention can be generally applied to implants or to medical devices which are released temporarily in the body and in which it is important that they be retractable into the corresponding delivery system. The retractability can play a role in repositioning, in particular of implants such as stents, or generally in the recovery of temporarily released medical devices.

The device according to FIG. 1 comprises a hollow body 10 with a longitudinal axis L. In the example according to FIG. 1, the hollow body 10 is rotationally symmetrical, in particular cylindrical. Other geometric shapes of the hollow body are possible, including shapes that are not rotationally symmetrical. The hollow body has a wall in the form of a braid 11 of wire elements 12. The wall is closed insofar as it surrounds and delimits the full circumference of the lumen of the hollow body. The wire elements 12 can be metal wires or plastic wires. The wire elements 12 are generally filaments or thread-shaped elements suitable for braiding. Possible materials for the wire elements are all customary materials suitable for implants or other medical devices to be inserted into a human hollow organ.

As can be seen in FIG. 1, the braid has a first series 13 of terminal meshes 14a, 14b, 14c, 14d arranged next to one another. The first series 13 of the terminal meshes 14a, 14b, 14c, 14d can also be seen clearly in FIG. 2. The terminal meshes 14a, 14b, 14c, 14d delimit an axial braid end 15. In the basket according to FIG. 1, the axial braid end 14 delimited by the terminal meshes 14a, 14b, 14c, 14d is open. The other braid end of the basket is closed. It is also possible, particularly in implants such as stents, to design the two axial braid ends in accordance with the invention. Generally, at least the retractable or proximal braid end should be designed in accordance with the invention.

The first series 13 with the terminal meshes 14a, 14b, 14c, 14d arranged next to one another continues in the direction of the longitudinal axis in the form of further series of meshes which together form the braid 11. The further series of meshes are produced or braided in a manner known per se. The terminal meshes 14a, 14b, 14c, 14d of the first series 13 have outer wire elements 12a to 12d, which together form the terminating edge 16 of the braid end 15. The outer wire elements 12a, 12b, 12c, 12d are therefore all arranged in the longitudinal direction L on the outside of the braid edge.

The course or the arrangement of the first and second axial components AK1, AK2 is shown in FIG. 3, likewise the course of the two circumferential components UK1, UK2. FIG. 3 shows the first and second section 16a of the terminating edge 16, with only the right-hand section 16b of the terminating edge 16 being shown completely. Of the left-hand section 16a of the terminating edge 16, the outer wire elements 12a, 12b, 12c, 12d brought together in the front terminal mesh 18 are shown. The other terminal meshes 14a, 14b, 14c correspond to the terminal meshes 14a, 14b, 14c shown in connection with the second section 16b (right-hand section in FIG. 3b). As can be seen from FIG. 3, each of the two terminating edges 16a, 16b has several outer wire elements 12a, 12b, 12c, 12d which together form a peripheral margin 15a of the terminating edge 16. In the illustrative embodiment according to FIG. 3, the peripheral margin is edgeless and thus facilitates the retraction of the braid end 15 into a delivery system. It is not impossible that the margin 15a has edges which extend in the distal direction, i.e. extend away from the user and thus do not protrude counter to the retraction direction. The retraction direction is shown by the arrow EZ in FIG. 3. It will also be seen there that the retraction direction EZ extends in the longitudinal direction L of the hollow body.

The first section 16a and also the (fully depicted) second section 16b of the terminating edge 16 are each composed of several outer wire elements 12a, 12b, 12c, 12d, which are arranged directly one after another along the terminating edge 16. In the present illustrative embodiment, the outer wire elements 12a, 12b, 12c, 12d are arranged flush in a line. It is also possible that the outer wire elements 12a, 12b, 12c, 12d at the terminating edge 16 are slightly offset outwardly or inwardly in relation to one another, in which case the resulting edges do not protrude counter to the retraction direction EZ.

The wire elements 12a, 12b, 12c, 12d are arranged directly one after another by means of the fact that, for each terminal mesh 14a, 14b, 14c, 14d, an inner wire element 12a′, 12b′, 12c′, 12d′ is delivered to the terminating edge and integrated into the latter. This results in the successive arrangement of additional outer wire elements 12a, 12b, 12c, 12d in the direction of the front terminal mesh 18. This principle applies to all the illustrative embodiments of this application, wherein the outer wire elements 12a, 12b, 12c, 12d are arranged substantially in a row along the terminating edge 16, whether or not they overlap in the course of the terminating edge 16.

FIG. 3 also shows the first axial component AK1 of the first section 16a (left-hand section in FIG. 3). Seen in the rotation direction ULR, the first axial component AK1 of the first section 16a extends in the proximal direction or retraction direction EZ. Seen in the same rotation direction ULR, the second axial component AK2 of the second section 16b extends counter to the retraction direction. This means that both axial components AK1, AK2 extend in the longitudinal direction L of the hollow body, and, seen in the same circumferential direction ULR, the axial components AK1, AK2 run in opposite directions.

By means of the uniform orientation of the outer wire elements 12a, 12b, 12c, 12d of the first section 16a and of the second section 16b with opposite axial components AK1, AK2, a shape of the terminating edge 16 is achieved which permits a retraction of the braid end into the delivery system. In this way, it is possible to form a smooth margin 15a of the terminating edge 16, which margin 15a slides without resistance into the delivery system. The orientation of the outer wire elements 12a, 12b, 12c, 12d with opposite axial components AK1, AK2 also permits the formation of edges that extend in the distal direction and thus likewise do not impede a retraction into the delivery system.

Moreover, the uniform orientation of the outer wire elements 12a, 12b, 12c, 12d of the respective section 16a, 16b does not exclude the possibility that the axial components of the individual outer wire elements 12a, 12b, 12c, 12d are of different sizes, such that a curved plane is obtained in which the terminating edge or the opening of the axial braid end lies. In the illustrative embodiment according to FIG. 3, the axial components AK1 and AK2 are each the same size. This results in a straight plane in which the terminating edge 16 lies, as shown in FIG. 1.

It will also be seen from FIG. 3 that the circumferential components UK1, UK2 of the outer wire elements 12a, 12b, 12c, 12d of the two sections 16a, 16b extend in the same direction, specifically seen in the circumferential direction UR of the hollow body. For the course of the circumferential direction UR, reference is made to the view according to FIG. 1, in which the circumferential direction is likewise indicated. The differences between the circumferential direction and the rotation direction, with respect to the wall and with respect to the margin 15a, are also clear from FIG. 1.

The course of the two axial components AK1, AK2 and of the two circumferential components UK1, UK2, as described with reference to FIG. 3, applies to all the illustrative embodiments of this application.

As is shown in FIG. 1, the terminating edge 16 can be arranged obliquely with respect to the longitudinal axis L. The angle of inclination can be adapted as desired, specifically via a variation of the braiding angle. The terminating edge 16 delimits a plane opening of the braid end 15. It is also possible that the terminating edge 16 has a curved contour, in particular a concave or convex contour. This applies to all the illustrative embodiments described in the application.

The structure of the terminating edge 16 can be seen particularly clearly in FIGS. 2 and 3. The design principle of the terminating edge 16 is that at least one first outer wire element 12a of a terminal mesh 14a extends in the area of another terminal mesh 14b, 14c, 14d. In the area of the other terminal mesh 14b, 14c, 14d, at least one second outer wire element 12b, 12c, 12d is provided, i.e. an additional wire element which extends together with the first outer wire element 12a along the terminating edge 16 and is connected to the first outer wire element 12a. This has the effect that the terminal meshes are interconnected and the terminating edge has a smooth and fixed margin. It is also possible to guide the wire elements 12a, 12b, 12c, 12d loosely, i.e. not connected, along the terminating edge.

The wires 12a, 12b, 12c, 12d do not have to be interconnected along the entire length. A slight twisting, which allows the wires 12a, 12b, 12c, 12d to be movable relative to one another, is also possible. For example, the wire 12a winds only once around the wire 12b at the level of the mesh 12b. This has the effect that the movement, in particular the compression, of both end loops 14a and 14b cannot take place independently of the other. Both loops engage in each other.

As is shown in FIG. 2, the outer (left-hand) first terminal mesh 14a, which lies opposite the section line S of the developed view, has a single outer wire element, which forms a first section of the terminating edge 16. It is also possible that the first terminal mesh 14a has more than one outer wire element 12a, for example 2, 3 or more outer wire elements 12a. Moreover, in the area of each new terminal mesh 14a, 14b, 14c, 14d, more than a single wire element can be added, e.g. 2 or 4 elements.

The outer wire elements 12a of the first terminal mesh 14a, or of the group of wire elements 12a of the first terminal mesh 14a, extends into the area of the adjoining second terminal mesh 14b. The second terminal mesh 14b has an additional second outer wire element 12b, which is connected to the first outer wire element 12a of the first terminal mesh. In this way, the terminating edge 16 has an increased thickness in the area of the second terminal mesh 14a, as is shown by the thicker line in FIG. 2. The two first and second outer wire elements 12a, 12b extend into the area of the downstream third terminal mesh 14c and are there connected to a third outer wire element 12c. The three outer wire elements 12a, 12b, 12c of the third terminal mesh 14c extend in the area of the fourth terminal mesh 14d and are there connected to a further outer wire element 12d, such that the fourth terminal mesh 14d has four outer wire elements. This principle can be applied to any desired number of terminal meshes. This applies to all the illustrative embodiments of this application.

Thus, the number of the outer wire elements generally increases in the circumferential direction and, in the case of an obliquely arranged terminating edge 16, toward the tip. In the illustrative embodiment according to FIG. 2, the first terminal mesh has one wire element, and the subsequent terminal meshes in the circumferential direction each have an additional wire element, such that the fourth terminal mesh 14d has four wire elements 12a, 12b, 12c, 12d, as is shown in FIG. 2. The increase in the number of the outer wire elements 12a, 12b, 12c, 12d per terminal mesh has the effect that the individual terminal meshes 14a, 14b, 14c, 14d each have a different number of outer wire elements 12a, 12b, 12c, 12d.

The wire elements of the respective terminal meshes 14a, 14b, 14c, 14d are interconnected, for example twisted or intertwined. Other connection possibilities, in particular integrally bonded or mechanical connecting means, are possible. This has the effect that the terminating edge 16 forms a stable margin.

As is shown in FIG. 2, the arrangement of the outer wire elements 12a, 12b, 12c, 12d along the terminating edge 16 is symmetrical with respect to the longitudinal axis L (see FIG. 1).

The terminal meshes can be formed, for example, by loops which overlap each other along the terminating edge 16 or which are offset along the terminating edge 16. In the illustrative embodiment according to FIG. 2, for example, the loop starting from the first terminal mesh 14a is the first loop, which extends as far as the front terminal mesh 18. The subsequent, second loop forms, together with the first loop, the first terminal mesh 14a (on the left in FIG. 2) and. To form the first terminal mesh 14a, the two loops are brought together at the location 17b and, in the area of the terminating edge 16, together form the first and second outer wire elements 12a, 12b. The second, third and fourth terminal meshes 14b, 14c, 14d and possible further terminal meshes are formed analogously. The above braiding configuration can also be clearly seen in FIG. 3, in the detail shown there. In particular, it can be clearly seen that the number of the outer wire elements 12a, 12b, 12c, 12d changes along the terminating edge 16, in particular increases toward the tip.

This means that the loops are brought together in a staggered arrangement at the successive locations 17b, 17c and 17d in the circumferential direction. The loops can be arranged symmetrically with respect to the longitudinal axis L such that

Other loop configurations are possible, resulting in a different number of outer wire elements 12a, 12b, 12c, 12d per mesh along the circumference of the terminating edge 16.

As can also be seen in FIGS. 1 and 2, the terminal meshes 14a, 14b, 14c, 14d have inner wire elements 12a′, 12b′, 12c′, 12d′ arranged in the braid 11. The inner wire elements 12a′, 12b′, 12c′, 12d′ are guided into the braid interior away from the terminating edge 16 and form the braid 11. As can be seen in FIGS. 2 and 3, the inner wire elements 12a′, 12b′, 12c′ and 12d′ are deflected and merge into the outer wire elements 12a, 12b, 12c, 12d. An inner wire element 12a′ and the associated outer wire element 12a thus belong to the same wire and form differently oriented sections of this wire.

The inner wire elements 12a′, 12b′, 12c′, 12d′ and outer wire elements 12a, 12b, 12c, 12d together form the loops described above.

At the transition of the inner wire elements 12a′, 12b′, 12c′ and 12d′ into the outer wire elements 12a, 12b, 12c, 12d according to FIGS. 1 to 3, the inner wire elements 12a′, 12b′, 12c′ and 12d′ change the direction of winding. This situation is depicted schematically in FIG. 3a. There, the terminating edge 16 is indicated by a broken line, and a braid-forming wire element 12x′ merges by deflection into an edge-forming wire element 12x. The expressions “edge-forming wire element” and “outer wire element” correspond to each other. Likewise, the expressions “inner wire element” and “braid-forming wire element” correspond to each other.

As can be seen in FIG. 3a, the braid-forming wire element 12x′ changes the direction of winding, and thus its circumferential direction, at the transition to the edge-forming wire element 12x. Specifically, the circumferential component UK of the wire element 12x′ extending in the circumferential direction UR of the wall changes. This means that the inner wires 12a′, 12b′, 12c′ and 12d′ or the braid-forming wires 12x′ extend clockwise for example, and the outer wire elements 12a, 12b, 12c, 12d or the edge-forming wire elements 12x extend counter clockwise. The longitudinal direction, or the component AK of the wire element 12x′ extending in the longitudinal direction L, remains constant.

It can also be seen in FIGS. 2 and 3 that the inner wire elements 12a′, 12b′, 12c′ and 12d′ and the outer wire elements 12a, 12b, 12c, 12d have the same braiding angle. The braiding angle is the angle that a wire element encloses together with the longitudinal axis. This applies to all the braid configurations described in the application.

As is shown in FIG. 2, the braid has a terminal mesh 18 arranged at the front in the longitudinal direction L of the hollow body 10, or a front terminal mesh 18. The terminal mesh 18 has outer wire elements 12a, 12b, 12c, 12d arranged in different axial directions with respect to the longitudinal direction L of the hollow body 10. The wire elements 12a, 12b, 12c, 12d thus have a change of axial direction in the area of the front terminal mesh 18.

In the illustrative embodiment according to FIG. 2, all the outer wire elements 12a, 12b, 12c, 12d are brought together in the front terminal mesh 18, for which reason the terminating edge 16 has the greatest diameter in the area of the front terminal mesh. The number of the outer wire elements 12a, 12b, 12c, 12d of the first section 16a and of the second section 16b in each case increases toward a front area 16c of the terminating edge 16 in the longitudinal direction L of the hollow body 10 and is at a maximum in the front area 16c, specifically in the area of the front terminal mesh 18.

The outer wire elements 12a, 12b, 12c, 12d can be part of a single group of wire elements, which group is deflected in the area of the front terminal mesh. The ends of the respective wire elements are in this case fixed in another area of the braid, in particular at the other end of the braid. Alternatively, the wire elements 12a, 12b, 12c, 12d coming from the one circumferential direction can form a first group of wire elements, and the wire elements 12a, 12b, 12c, 12d coming from the other circumferential direction can form a second group of wire elements. The two groups of wire elements are interconnected in the area of the tip of the front terminal mesh 18.

Particularly in the case where the same wires 12a, 12b, 12c, 12d are deflected as a whole group, they are not deformed sharp in the area of the front terminal mesh 18, as shown schematically in the drawings. Instead, the end of the mesh has a rounding. If different wire groups are brought together, the groups can extend tangentially to one another in the distal direction.

In the illustrative embodiment according to FIGS. 1 to 3, the last wire of one (developed) side is singulated and no longer has to be deflected. Instead, it extends onward in a straight line and becomes the inner (braid-forming) wire element. The last wire can also be a wire bundle of several wires. It is also possible that two wires branch off at each loop. It is also possible for more than two wires to be branched off. This results in a configuration in which the wires in the different sections are twisted, e.g. 8 wires, 6 wires, 4 wires, 2 wires. Other configurations are also possible.

The wires are twisted in the area of the contour of the terminating edge 16, as a result of which a compact configuration is obtained. This permits the insertion of the device into small catheter lumens. The branching-off (or delivery) of each individual wire element can be such that the wire element merges smoothly into the twist. This is possible, for example, through the design of the twist and the exact position of the branch-off. It is also possible that the wire elements are not twisted but braided. This increases the stability of the wire strands in the area of the terminating edge 16. A combination of twisting and braiding is also possible.

Another illustrative embodiment of a device with another braid configuration is shown in FIG. 4. In principle, the braid configuration according to FIG. 4 differs from the braid configuration according to FIGS. 1 to 3 in that the direction of the axial component AK of the wires changes at the transition of the outer wire elements 12a, 12b, 12c, 12d into the inner wire elements 12a′, 12b′, 12c′ and 12d′. The direction of winding or the circumferential component UK remains the same at the transition. This situation is shown in FIG. 3b. It will be seen in FIG. 3b that the axial component AK of the braid-forming wire element 12x′ extends in an opposite direction to the axial component AK of the edge-forming wire element 12x.

In the braid combination according to FIG. 4, the number of the outer wire elements 12a, 12b, 12c, 12d decreases toward the tip, or toward the front terminal mesh 18, and is at a minimum there, in contrast to the configuration according to FIGS. 1 to 3, in which the number of the outer wire elements increases toward the tip. Otherwise, the design principle is the same as in the example according to FIGS. 1 to 3. Also in the illustrative embodiment according to FIG. 4, staggered loops are provided, which form the terminal meshes 14a, 14b, 14c, 14d and are brought together at different locations 14a, 14b, 14c, 14d arranged in succession along the terminating edge 16. At the transition from one loop to the next loop, a wire of the wire group is deflected, specifically in the opposite axial direction (FIG. 3b).

The last loop consists of a single wire element 12a and is arranged in the area of the front terminal mesh 18. It will also be seen from FIG. 4 that the last wire group, which comprises all the outer wire elements, in particular the four wire elements of the example according to FIG. 4, is continued in the braid. This wire strand 21 arranged away from the front terminal mesh 18 is integrated into the braid and has the effect that the braid is stabilized along its entire length. It is particularly advantageous if the wire elements of the wire strand are twisted, particularly in the same direction of winding. Other wire combinations are possible, in particular a different number of wires of the wire strand.

FIGS. 6a, 6b show two further illustrative embodiments of the device according to the invention, in which the outer wire elements 12a, 12b, 12c, 12d of the front terminal mesh 18 form a continuation 19, which extends beyond the contour of the terminating edge 16. The continuation 19 has a functional element, in particular a connecting element 20, which is designed as a loop, for example. The loop can be used to connect the device to an actuation element, in particular to a guide wire of a delivery system. Otherwise, the braid configuration of the illustrative embodiments according to FIGS. 6a, 6b corresponds to that of the above-described illustrative embodiments according to FIGS. 1 to 3 and 5.

Generally, the wires in the area of the continuation 19, in particular the outer wire elements 12a, 12b, 12c, 12d, can be twisted together regardless of whether the wires in the terminating edge 16 are twisted or loose. The twisted arrangement of the wire elements in the area of the continuation 19 is therefore disclosed in connection with all wire configurations, in particular with twisted, loose, braided, mechanically connected or integrally bonded wire configurations. The twisting in the area of the continuation 19 increases the stability. Instead of the loop, the wires can have free ends. A sleeve, or generally a profiled endpiece, can be secured on the twisted arrangement in the area of the continuation 19. The sleeve, or the profiled endpiece, can be made of an X-ray-visible material, for example platinum. The connection between the sleeve, or the profiled endpiece, and the wires in the area of the continuation 19 can be in the form of a welded connection, a crimped connection, an adhesively bonded connection or another kind of mechanical connection. For example, the wire ends surrounded by the sleeve can, together with the sleeve, be welded at the front face, in particular by a semi-spherical weld. The front faces of the free ends can also be welded at the front face without a sleeve.

The continuation 19 preferably extends parallel to the longitudinal axis of the braid. This is not obligatory. The straight continuation can protrude radially outward. A radially outwardly rounded shape of the continuation 19 is likewise possible. The continuation 19 preferably extends in the same plane in which the oblique terminating edge 16 is arranged. The plane corresponds generally to a curved and oblique sectional face through a circular cylindrical hollow body. In this way, a gentle transition between the terminating edge 16 and the continuation 19 is formed. The cylinder jacket surface can be curved outward (flaring).

In all of the disclosed illustrative embodiments, the braid can have at least 8, 12, 16, 24, 32, 36, 40, 48, 60, 72, 84, 96 wires, this corresponding to the number of wires intersecting a plane perpendicular to the braid axis. In the case of closed loops, the actual number of wires is halved. The braiding angle, in particular the braiding angle of the terminating edge, is at least 20° 30° 40° 45° 50° 60° 70° 80°. In this way, the oval area is shortened. The braiding angle, in particular the braiding angle of the terminating edge, is at most 80° 70° 60° 50° 45° 40° 30° 20°. The gentle incline thereby obtained makes the insertion into the catheter easier.

The system is insertable into a catheter with an internal diameter of at most 2 mm, in particular at most 1.8 mm, in particular at most 1.5 mm, in particular at most 1.3 mm, in particular at most 1.1 mm, in particular at most 1.0 mm, in particular at most 0.9 mm, in particular at most 0.8 mm, in particular at most 0.7 mm, in particular at most 0.6 mm, in particular at most 0.5 mm, in particular at most 0.4 mm, in particular at most 0.3 mm, in particular at most 0.2 mm.

The braid can have wires with differing wall thickness.

The braid can be covered, preferably with PU. It can be partially covered. For example, the basket for removing a clot can be covered only in the distal area.

The wires can be made from nitinol, cobalt/chromium alloy or nitinol wires with a platinum core.

The coupling of the device according to FIGS. 6a and 6b to the guide wire of a catheter is shown in FIGS. 7 and 8. The delivery system is provided generally with the reference sign 30 and has a catheter 31 in which an actuation element, in particular a guide wire 32, is arranged in the longitudinal direction. The guide wire comprises a decoupling mechanism 33, which has a proximal end 33a. The proximal end 33a of the decoupling mechanism 33 is connected fixedly to the guide wire 32. This is followed in the distal direction by a release element 33b, which is connected to the proximal end 33a and is pretensioned in the radial direction in the catheter 31. When the release element 33b is freed, as is shown in FIG. 7, it deploys in the radial direction, thereby shortening the decoupling mechanism 33 in the longitudinal axial direction. The release element 33b is connected to the intermediate piece 34, in particular to an intermediate piece 34 arranged in a longitudinally displaceable manner on the guide wire 32. The intermediate piece 34 is connected fixedly to a holding means 35, for example a sleeve. The intermediate piece 34 and the sleeve 35 can be actuated by a movement of the release element 33b; they can in particular be drawn back along the guide wire 32 in the longitudinal direction.

The holding means 35 specifically comprises, as shown in FIG. 8, a sleeve 35b, which is arranged so as to be longitudinally displaceable along the guide wire, and a pin 35a or a locking element, which is connected fixedly to the guide wire 32. The loop 20 of the continuation 19 of the basket engages around the pin 35a. By drawing the sleeve 35b back, the locking of the pin 35a is canceled, or the pin is freed, such that the loop 20 is moved radially outward by the radial expansion of the basket. The basket is thus decoupled from the delivery system.

The delivery system is described in more detail in the application entitled “Delivery system for a medical functional element”, which was filed by the applicant on the same day. The content of said application is incorporated in full into the present application by reference, since the example according to FIGS. 6a, 6b can be combined with the delivery system disclosed therein.

FIG. 9 shows an example of a possible use of the basket according to the invention or generally of the device according to the invention. It will be seen from FIG. 9 that the device according to the invention is arranged in a blood vessel 50 for the purpose of removing a clot or concretion 40. After the clot has been drawn into the basket, the continuation 19 allows the smooth terminating edge 16 to be drawn back easily into the catheter.

Further illustrative embodiments with different braid configurations are shown in FIGS. 10 to 13 in which, seen in the rotation direction ULR of the margin 15a, the outer wire elements 12a to 12g or 12a to 12d each have different axial components AK1 and AK2, i.e. axial components oriented in different directions, and specifically in both sections 16a, 16b of the terminating edge 16.

In the illustrative embodiment according to FIG. 10, the first section 16a and the second section 16b of the terminating edge 16 each have a front area 16c, a rear area 16d and, arranged between these, an intermediate area 16e. As is shown in FIG. 10, the front area 16c is located at the front in the longitudinal direction L of the hollow body, i.e. in a proximal area of the terminating edge 16. The rear area 16d is located in a distal area of the terminating edge 16. With the terminating edge 16 arranged at an incline, the rear area 16d is adjoined by the closed and in particular cylindrical wall of the braid. The intermediate area 16e is located between the front area 16c and rear area 16d. Starting from the intermediate area 16e, the number of the outer wire elements 12a, 12b, 12c, 12d in each case decreases toward the front area 16c and rear area 16d. The number of the outer wire elements 12a, 12b, 12c, 12d is at a minimum in the front area 16c and rear area 16d. The number of the outer wire elements 12a, 12b, 12c, 12d is at a maximum in the intermediate area. In the present illustrative embodiment according to FIG. 10, the intermediate area 16e is arranged symmetrically between the front area 16c and rear area 16d. It is also possible to arrange the intermediate area 16e closer to the front area 16c or closer to the rear area 16d.

The wire path of a loop is indicated in FIG. 10 by arrows. The arrow I designates the course of a braid-forming wire element 12x′, which is deflected in the terminating edge 16 and extends along the terminating edge 16 in arrow direction II. There, the braid-forming wire element 12x′ merges into the edge-forming wire element 12x or the outer wire element. At a distance of four meshes from the braid-forming wire element 12x′ (arrow I), the edge-forming wire element 12x is deflected back again into the braid and merges again into a braid-forming wire element 12x′, which is designated by the arrow III. The arrows I, II and III indicate the course of the loop. The further loops are arranged along the terminating edge 16, in each case offset by one terminal mesh, and otherwise correspond to the course of the above-described loop. This results in the position of the intermediate area 16e, in which the number of the outer wire elements 12a, 12b, 12c, 12d is at a maximum. The reason for this is that all the loops overlap in the intermediate area 16e.

The loops of the first section 16a are arranged correspondingly.

It will also be seen from FIG. 10 that the outer wire element 12a of the terminal mesh 14a farthest from the front terminal mesh 18 is not deflected, but instead merges directly into the braid. This is illustrated by the wire designations a, b in FIG. 10. If the developed view according to FIG. 10 is closed to form a cylindrical hollow body, the broken line b (right-hand side) lies on the solid line b (left-hand side of the developed view).

This applies to all the embodiments of this application.

The illustrative embodiment according to FIG. 11 is of similar construction to the illustrative embodiment according to FIG. 10 and comprises several loops nested one inside another, as is illustrated by the dotted and solid arrows. As is shown in FIG. 11, the loops of the first section 16a and the loops of the second section 16b are increasingly smaller from the outside inward. The outer loop (dotted arrows) comprises five terminal meshes, for example. The inner loop (solid arrows) arranged in the outer loop comprises three terminal meshes. The smallest loop, which is arranged inside the middle loop (solid arrows), comprises one terminal mesh. In this way, the loops are each inwardly offset by one terminal mesh on both sides. This arrangement also has an intermediate area 16e, as has been described in detail in connection with FIG. 10.

Another illustrative embodiment is shown in FIG. 12, in which the terminating edge 16 of each terminal mesh 14a, 14b, 14c, 14d comprises only one wire. It is also possible that each terminal mesh comprises several wires, in which case the outer wires 12a, 12b, 12c, 12d do not overlap each other, but instead are in each case limited to the associated terminal mesh 14a, 14b, 14c, 14d. As can be seen in FIG. 12, the inner wire elements 12a′, 12b′, 12c′, 12d′ form inner mesh limits 36a, 36b which, starting from the terminating edge 16, extend into the braid interior. The inner mesh limits 36a directly adjoin the terminating edge 16 or the associated outer wire elements 12a, 12b, 12c, 12d. The further inner mesh limit 36b extends parallel to the terminating edge 16, generally at a distance therefrom, and arranged opposite it. In order to fix the terminal meshes 14a, 14b, 14c, 14d, the inner mesh limits 36a and/or 36b are at least in part interconnected. This means that the inner wire elements 12a, 12b, 12c, 12d are in part bound together in the inner area of the meshes, preferably twisted. At the transition of the outer wire elements 12a, 12b, 12c, 12d to the inner wire elements 12a′, 12b′, 12c′, 12d′, there is always a change of direction of the wire in the circumferential direction, with one exception. Only in the wire of the last terminal mesh, i.e. of the terminal mesh 14a, adjoined by the closed wall, in particular the cylindrical wall of the hollow body, there is no change of direction of the wire in the circumferential direction. The corresponding wire is not turned back, but is instead continued into the braid, as has been described above in connection with FIG. 10. The embodiment according to FIG. 12 can be combined with the embodiments described above. The illustrative embodiment according to FIG. 12 also shows that the individual terminal meshes in the area of the outer wire elements 12a, 12b, 12c, 12d can be slightly offset in relation to one another. Here, the terminal meshes are offset successively further outward in the retraction direction, which results overall in an inwardly set back terminating edge 16, as is shown in FIG. 12, which can be drawn into a delivery system without jamming.

In the illustrative embodiment according to FIG. 13, the examples according to FIGS. 11 and 12 are combined with each other. This results in a braid configuration in which the terminal meshes 14a, 14b, 14c, 14d of the first section 16a and of the second section 16b each have at least two loops 37a, 37b, which are arranged next to each other along the terminating edge 16. A third loop 37c overlaps the two first and second loops 37a, 37b. The wire path of the first loop 37a is shown by the dotted arrows. In this connection, it is once again clear that the outer wire element of the last terminal mesh 14a is not turned back, but is instead continued in the braid. The last terminal mesh 14a directly adjoins the closed wall or cylindrical wall of the braid. In other words, the last terminal mesh 14a is located at the apex of the terminating edge 16. The second adjoining loop 37b is indicated by the dot-and-dash arrows, which illustrate the wire path of the second loop 37b. It is clear from the wire path that the first and second loops do not overlap each other, but are instead arranged adjacent to each other along the terminating edge 16. The two first and second loops 37a, 37b are overlapped by a third loop 37c, of which the wire path is indicated by the solid arrows. The third loop 37c overlaps the two first and second loops 37a, 37b in each case by one terminal mesh. Other degrees of overlap are possible. The overlap can be symmetrical, as in FIG. 13. It is also possible to provide asymmetrical overlaps. The loop overlap in FIG. 13 has the effect that some wires or wire sections extend inside the braid, i.e. not in the area of the terminating edge 16. These wires extend in parallel and can be connected to one another, for example by twisting or other forms of connection. Other wire sections, particularly in the area of the third loop 37c, extend together in the area of the terminating edge 16 and can likewise be connected or be arranged loosely.

It is thus clear that the above-described illustrative embodiments permit different combinations, which allow a variable design of the braid, with the result that the mechanical properties, for example the radial force, flexibility and mesh fineness, can be very precisely adapted.

In all of the illustrative embodiments, the device in the expanded state can be funnel-shaped (flared) at the axial end, preferably in the area of the terminating edge 16. This means that the lattice braid is widened in the direction of the axial end, i.e. has a preferably continuously increasing cross-sectional diameter. The funnel-shaped widening is preferably arranged at a distal end of the lattice braid. The distal end corresponds to the axial end of the lattice structure which, upon release of the device from a catheter, is first to leave the catheter or the catheter tip. The distal end is thus arranged away from the user, whereas a proximal end of the lattice structure or of the device is directed toward the user. The funnel-shaped widening at the distal end of the lattice structure has the effect of facilitating the expansion of the device upon release from a delivery system. A widening at the distal axial end of the medical device is advantageous in particular in recanalization systems or baskets, particularly clot catchers or filters, which deploy distally in the hollow organ. Alternatively, the proximal axial end of the lattice braid, i.e. the axial end near the user during use, can have a funnel-shaped, in particular flared structure. This embodiment is suitable for devices which are designed as clot catchers or baskets with a proximally expandable axial end. It is also possible for both the proximal axial end and also the distal axial end of the lattice braid to have a funnel shape. This embodiment is advantageous, for example, in a device which is designed as a permanent implant or stent, in particular a stenosis stent or aneurysm stent. It is generally preferable if the funnel-shaped end forms an access to a cavity inside the lattice braid. A further axial end of the medical device can be closed or can likewise have a funnel shape.

As was explained at the outset, the outer wire elements 12a are formed by deflection, i.e. by a change in the direction of winding of the inner wire elements 12a′. An outer and inner wire element 12a, 12a′ thus form a one-part wire element 12A, of which the free end is arranged, and optionally fixed, in the area of the tip of the terminating edge 16 or at the axial braid end 15. The inner wire element 12a′ forms the braid-forming section of the one-part wire element 12A. The outer wire element 12a is formed by deflection of the inner wire element 12a′ and is integrated in the terminating edge 16. The outer wire element 12a forms the edge-forming section of the one-part wire element 12A. The inner wire element 12a′ thus corresponds to the non-deflected wire element 12A, and the outer wire element 12a corresponds to the deflected wire element 12A. This applies to all the wire elements, wherein the first wire element 12A, 12 comprise outer wire element 12a, which are guided into the terminating edge 16 without changing the spiral direction. In wires that are not deflected, 12a corresponds to the continuation of 12a′ in the terminating edge 16. The following explanations are therefore disclosed in connection with all of the illustrative embodiments and generally in connection with the invention.

FIGS. 14a, 14b, 15a, 15b and 16 show a medical device, in particular an implant for removal of concretions from hollow organs of the body, with a compressible and expandable lattice braid 11 that comprises several wire elements 12A, 12B, 12C, 12D with a first spiral direction and several wire elements 12E, 12F, 12G, 12H with a second spiral direction, which are each wound about a common longitudinal axis and intersect each other to form meshes. The lattice braid 11 has a peripheral terminating edge 16 with a first section 16a and second section 16b, wherein the first section 16a and second section 16b extend in different spiral directions along the terminating edge 16. The two sections 16a, 16b meet at the tip of the terminating edge 16 or at the braid end 15. Each of the two sections 16a, 16b is formed in each case by at least two wire elements, of which at least one wire element at the transition from the lattice braid 11 to the terminating edge 16 is deflected at a deflection location in such a way that the deflected wire element extends along the terminating edge 16 in another spiral direction than inside the lattice braid 11. The deflected wire element thus experiences a change in the spiral direction at the transition from the braid 11 to the terminating edge 16. The terminating edge 16 is smooth and runs round continuously, i.e. without projections.

The wire elements 12A, 12B, 12C, 12D with the first spiral direction are arranged axially symmetrically with respect to the wire elements 12E, 12F, 12G, 12H with the second spiral direction, as is shown in FIG. 14a. The comments regarding the wire elements 12A, 12B, 12C, 12D with the first spiral direction are also disclosed in connection with the wire elements 12E, 12F, 12G, 12H with the second spiral direction. The wire element which is farthest from the braid end 15 or from the tip of the terminating edge 16, and which opens into the terminating edge 16 in the area of the apex 39, is designated as first wire element 12A. The corresponding wire element with the second spiral direction is designated by 12H. The first wire element 12A is deflected on the first pin 23 and merges, with a change of the spiral direction, into the terminating edge 16, specifically into the first section 16a of the terminating edge 16. The spiral direction of the first section 16a corresponds to the second spiral direction of the wire elements 12E, 12F, 12G, 12H. Thus, at the transition from the braid 11 to the terminating edge 16, the first wire element 12A changes from the first spiral direction to the second spiral direction.

Likewise, at the transition from the braid 11 to the terminating edge 16, specifically at the transition to the second section 16b, the first wire element 12H with the second spiral direction changes the spiral direction in such a way that the first wire element 12H in the area of the terminating edge 16 extends in the first spiral direction. Thus, with respect to the same rotation direction ULR of the margin 15a (see FIG. 2 for example), the two first wire elements 12A, 12H have opposite axial components AK1, AK2, as is described in more detail in connection with FIGS. 2, 3, 3a and 3b. This applies to all the wire elements that merge into the terminating edge 16, in particular to the second wire elements 12B, 12G, the third wire elements 12C, 12F, and the fourth wire elements 12D, 12E, and also to any further wire elements integrated in the first and second sections 16a, 16b, as shown in FIG. 16.

Because of the cut-open view according to FIG. 14a, the last wire element 12E, for example, which has the second spiral direction and which opens into the second section 16b of the terminating edge 16 near the tip of the terminating edge 16 or of the braid end 15, terminates at the section line S. By contrast, in the unopened three-dimensional form of the device, the last wire element 12E continues its spiral shape on the opposite side of the view according to FIG. 14a in the distal direction, i.e. downward in the plane of the drawing, as is shown by the course of the wire element 12E. This applies correspondingly to all the wire elements 12A, 12B, 12C, 12D, 12E, 12F, 12G, 12H.

The wire elements 12A, 12B, 12C, 12D, 12E, 12F, 12G, 12H, coming from the braid 11, open at different levels into the terminating edge 16, in each case at the same angle. Some, in particular half, of the wire elements 12A, 12B, 12C, 12D open into the first section 16a of the terminating edge 16, and some, in particular the other half, of the wire elements 12E, 12F, 12G, 12H open into the second section 16b of the terminating edge 16. The two sections 16a, 16b form symmetrical halves or, generally, segments of the terminating edge 16.

In connection with all the illustrative embodiments and, generally, in connection with the invention, it is disclosed that the wire elements 12A, 12B, 12C, 12D, 12E, 12F, 12G, 12H, which merge into the terminating edge 16, or the inner wire elements 12a′, 12b′, 12c′, 12d′ in the area of the terminating edge 16 experience an abrupt or discontinuous change in the spiral direction or the circumferential direction. The wire elements 12A, 12B, 12C, 12D, 12E, 12F, 12G, 12H thus form a defined angle at the transition to the terminating edge 16, for example an angle of ca. 90°. Other angles are possible, particularly in a range of 90° to 60°, preferably 70°. The braiding angle can be 45° to 60°, in particular ca. 55°. For example, the braiding angle of 45° corresponds to the transition angle of 90°. The other angles behave accordingly.

By means of the same orientation of the wire elements 12A, 12B, 12C, 12D, 12E, 12F, 12G, 12H at the transition to the terminating edge 16, i.e. by means of the deflection at the same angle, all the wire elements 12A, 12B, 12C, 12D, 12E, 12F, 12G, 12H have the same length. The wire elements 12A, 12B, 12C, 12D, 12E, 12F, 12G, 12H thus open into the terminating edge 16 at different discrete locations and at the same angle. For example, the first wire element 12A extends along the entire length of the first section 16a. The last wire element 12D extends only along one mesh in the area of the terminating edge 16, but is thus correspondingly longer in the braid, such that the same or at least approximately the same wire length is obtained. The same applies to the other wire elements.

The fact that the wire elements 12A, 12B, 12C, 12D, 12E, 12F, 12G, 12H are of substantially the same length means that the crimpability of the braid is improved, since no distortions, or at least no significant distortions, arise during the compression.

All of the wire elements 12A, 12B, 12C, 12D, 12E, 12F, 12G, 12H, or all of the inner wire elements 12a′, 12b′, 12c′, 12d′, can be deflected discontinuously into the terminating edge 16. This applies both to inner wire elements 12′ which are composed of individual wires or which are each composed of wire bundles. It is also possible that all of the wire elements 12B, 12C, 12D, 12E, 12F, 12G, or all of the inner wire elements 12b′, 12c′, 12d′, i.e. except for the first wire elements 12A, 12H or except for the first inner wire elements 12a′, are deflected completely.

In the case of the first wire elements 12A, 12H opening into the terminating edge in the area of the apex 39, it is thus necessary to distinguish between three possibilities. The two first wire elements 12A, 12H are deflected in the area of the apex and, on entering the terminating edge, each change the spiral direction, as is shown in FIG. 14a. The abrupt change in the spiral direction results in a gap at the apex 39. The gap corresponds to an open cell 38, which directly adjoins the apex of the terminating edge 16. The two halves of the cell 38 are shown in the developed view according to FIG. 2 and FIG. 14a. The open cell 38 does not impede the retractability of the braid, since the terminating edge 16 is edgeless, except for the apex, or has an edgeless or continuous course. This embodiment of the invention is covered by the term “edgeless”.

The two first wire elements 12A, 12H can each be composed of individual wires or of wire bundles having several individual wires or of a corresponding combination.

Alternatively, the two first wire elements 12A, 12H in the area of the apex can maintain their spiral direction, i.e. are not deflected, as shown in FIG. 14b. The first wire element 12H, terminating at the section line S in the cut-open view, continues on the opposite (left-hand) side of the braid and merges into the terminating edge 16 without changing the spiral direction or circumferential direction. Thus, the two first wire elements 12A, 12H intersect each other at the apex 39. No gap is formed. Here too, the two first wire elements 12A, 12H can each be composed of individual wires or of wire bundles having several individual wires or of a corresponding combination.

If the two first wire elements 12A, 12H are each composed of wire bundles having several individual wires, some of the individual wires of a bundle can be deflected. The others can maintain the spiral direction. Thus, the individual wires of one first wire element 12A, 12H are divided at the apex 39 and some of them merge into the first section 16a and some into the second section 16b. Here too, no gap is formed. This applies to both first wire elements 12A, 12H. Otherwise they correspond to the aforementioned illustrative embodiments.

Therefore, for the two first wire elements 12A, 12H, the following combination possibilities exist: All of the individual wires are each deflected or are each guided into the terminating edge 16 without changing direction. All the individual wires of one wire element 12A are deflected, and all the individual wires of the other wire element 12H are not deflected. All of the wires open into the same section 16a, 16b. All the individual wires of one wire element 12A are deflected or not deflected. The individual wires of the other wire element 12H are split and in part deflected or undeflected.

The illustrative embodiments according to FIGS. 15a, 15b are based on the illustrative embodiments according to FIGS. 14a, 14b. In addition, in the illustrative embodiments according to FIGS. 15a, 15b, provision is made that the wire elements 12A, 12B, 12C, 12D, 12E, 12F, 12G, 12H each comprise individual wires. The individual wires of the respective wire elements 12A, 12B, 12C, 12D, 12E, 12F, 12G, 12H are laterally spaced apart in the braid 11. At the terminating edge 16, the individual wires of the respective wire elements 12A, 12B, 12C, 12D, 12E, 12F, 12G, 12H come together and are deflected in union into the terminating edge 16. The deflection locations distributed along the terminating edge 16, in particular distributed at equal intervals, are defined by the deflection pins 23, which do not belong to the device but to the manufacturing tool. The arrangement of the deflection pins 23 defines the form of the terminating edge 16.

The distance between the individual wires of the respective wire elements 12A, 12B, 12C, 12D, 12E, 12F, 12G, 12H decreases at least in the area of the last mesh, in particular in the area of the last two meshes before the terminating edge 16, until the individual wires touch each other in the area of the deflection location and continue together along the terminating edge 16. As the distance decreases, the individual wires come closer to each other tangentially. The tangential convergence is limited to the individual wires of each wire element. The deflection into the terminating edge 16 takes place discontinuously. Thus, on account of the tangential convergence before the terminating edge 16, there is only negligible distortion during crimping. In the example according to FIG. 15a, as in the example according to FIG. 14a, the individual wires of the two first wire elements 12A, 12H are deflected, such that a gap forms in the area of the apex 39.

The distortion of the braid 11 is also limited by the fact that the number of the individual wires per wire element 12A, 12B, 12C, 12D, 12E, 12F, 12G, 12H is limited in particular to a maximum of 6 individual wires, in particular a maximum of 5, in particular a maximum of 4, in particular a maximum of 3, in particular a maximum of 2.

On each pin there are two wires, which converge in the braid shortly before the pin. In each case, however, there are only two wires per pin. The distortion is thereby reduced. The main thing is that the number of wires converging tangentially is reduced, with a gradual or abrupt change of the course. The ratio of the number of individual wires per wire element to the total number of wires is at most 25%, in particular at most 20%, in particular at most 15%, in particular at most 10%, in particular at most 8%, in particular at most 6%, in particular at most 5%, in particular at most 4%.

The advantage of the multiple wire configuration is that, in a very fine wire braid, on account of the large number of individual wires, a correspondingly close positioning of the pins 23 of the manufacturing mandrel is limited by the maximum pin diameter. By means of the formation of wire bundles, the terminating edge 16 can be formed with a sufficiently large number of deflection pins 23, wherein the fine mesh of the braid is maintained. The individual wires of the wire bundles can be arranged in parallel, i.e. untwisted, next to each other. The individual wires can also be twisted or braided. The parallel individual wires of the respective wire elements 12A, 12B, 12C, 12D, 12E, 12F, 12G, 12H can form loose wire bundles. A loose wire bundle is understood as a wire bundle in which the individual wires are not fixed to each other, such that they are movable relative to one another, at least compared to a twisted or braided wire configuration in which the relative movement of the individual wires is limited.

The nature of the wire path toward the pin 23 is determined by the braiding technique. This can be, for example, 1 over 1, 1 over 2 or similar, and ensures that the convergence of the wires takes place just before the pin. This also stabilizes the braid. The braiding technique 2 over 2, 2 over 4, 4 over 4 or similar has the effect that the convergence is gentler, or the area of convergence longer. The braiding technique can change. For example, the braiding technique 1 over 1 can be present in the braid, and, closer to the terminating edge 16, the braiding technique 2 over 2, 2 over 4, 4 over 4 or similar may be present.

In the illustrative embodiment according to FIG. 15b, the individual wires of the two first wire elements 12A, 12H are not deflected (cf. FIG. 14b), but instead open into the respective edge section 16a, 16b without changing the spiral direction. The individual wires therefore intersect each other in the area of the apex 39. Therefore, no deflection pins 23 are shown in this area. The deflection pins may be present in this area.

The tangential convergence of the individual wires of the two first wire elements 12A, 12H takes place inter alia along the terminating edge 16, and specifically approximately over the edge length of at most two meshes. In the further course of the terminating edge 16, the individual wires of the two first wire elements 12A, 12H touch. If there are 2 individual wires per wire element 12A, 12H, the distortion is unproblematic. Here too, it is possible to have up to 6 individual wires, in particular a maximum of 5, in particular a maximum of 4, in particular a maximum of 3, in particular a maximum of 2. The tangential convergence is limited, for example, to 2 wires. The distortion is thus likewise limited. That wire at a certain distance from the terminating edge 16 is also arranged outside the terminating edge and therefore does not belong to the braid 11. The distortion is further limited for this reason too. The terminating edge 16 is clearly defined by the course of the pins 23 in the direction of the braid.

Another example of a multiple wire configuration with at least 2 wire elements, in particular at least 3 or at least 4, each having several individual wires, in particular having two individual wires, is shown in FIG. 16. All the wire elements or individual wires which are located inside the area between apex 39 and tip 15 and represent inner wires, i.e. do not already belong to the terminating edge 16, experience an abrupt change of the spiral direction in the direction of the respective terminating edge section 16a, 16b.

All the features of the examples according to FIGS. 14a, 14b, 15a, 15b and 16 are disclosed and claimed in connection with all the other illustrative embodiments.

To carry out the method for producing a device according to FIGS. 1 to 9, a braiding mandrel is used on the periphery of which the pins shown in FIGS. 2, 3 and 4 are arranged. The pins 23 serve to change the direction of winding and/or the direction of the axial component of the wire elements at the transition from the braid-forming to the edge-forming wire elements. In FIG. 3, the pins 23 are shown only on one side of the inner wire elements 12′. As is shown in FIG. 2, pins 23 are in practice provided on both sides. As will be clear to a person skilled in the art, the pin 23 arranged on the inside with respect to the deflected wire element 12′ is the pin needed for the deflection of the inner wire element 12′. In an alternative arrangement of the pins differing from the arrangement according to FIG. 2, it suffices if one pin 23 is in each case arranged on the inside of the deflected inner wire element 12′. To this end, in the illustrative embodiment according to FIG. 3, the pins 23 shown are each to be transposed to the other side (left-hand side in FIG. 3) of the inner wire elements 12′.

If, in the illustrative embodiment according to FIG. 2, all the inner wire elements 12′ are deflected, that is to say also the first inner wire element 12a′, a further pin 23 is in each case arranged in the first terminal mesh 14a on the inside of the deflected first inner wire element 12a′. In the illustrative embodiment according to FIG. 16, further deflection pins can be provided directly in the area of the tip and/or laterally from the continuation 19 and limit the twisting of the continuation 19. In a radial deflection of the tip or of the continuation 19, a mandrel with a corresponding widening can be used.

The following description of a method for producing the device according to the invention applies to all of the illustrative embodiments.

To produce a device according to the invention, in particular according to one of the illustrative embodiments explained above, a braiding machine is generally used. With the braiding machine, the wire elements are automatically braided to form the braid 11. The braiding machine comprises reels, onto each of which a wire element is wound. The reels are guided in a circle around a common braiding point, and the wire elements are unwound from the reels. During the braiding operation, the reels are moved further in the radial direction. This means that the reels are moved toward the braiding point or away from the braiding point. For this purpose, the reels are arranged on reel carriers or bobbins. In particular, two reels can be arranged on each bobbin. Several groups of bobbins are preferably provided, wherein at least a first group of bobbins rotates counter to a second group of bobbins around the braiding point. In doing so, the bobbins travel in a serpentine or zigzagging manner. The direction of rotation of the bobbin groups determines the direction of winding of the wire elements in the tubular braid. At the axial end, which is intended to have a particularly smooth edge, the wire elements are accordingly deflected by means of the direction of rotation of the bobbins being reversed. In order to twist the wire elements in the terminating edge, two reels are rotated about a common twist axis, or the bobbin carrying the two reels is rotated about the bobbin axis. The rotation of the reels about the common twist axis can be superposed by the rotation of the bobbin groups about the braid axis or the braiding point.

With the braiding machines known per se, it is possible to produce tubular lattice structures and also flat braids. A flat braid can then be bent into the tube shape and connected, for example welded, at the contiguous longitudinal edges. The braid 11 can be braided both in a closed structure and also in an open structure. In the closed structure, the braiding operation is started from the terminal mesh 18. The first section 16a and the second section 16b of the terminating edge 16 are produced at the same time. Alternatively, the lattice structure or the braid 11 can be produced as an open structure. The braid 11 is produced continuously and divided into desired length sections, which each have open wire ends. The open wire ends are then deflected and twisted, for example by hand, in order to form the terminating edge 16. It is also possible for the twisting to already take place in the braiding machine, such that, after the continuous braid strand has been divided, the terminating edge 16 is produced by suitable arrangement of the twisted sections. The twisted sections are arranged such that a continuously smooth terminating edge 16 is formed.

It is also possible for the device according to the invention, in particular according to one of the above-described illustrative embodiments, to be produced entirely by hand.

### LIST OF REFERENCE SIGNS

S section line

L longitudinal axis

LR longitudinal direction

UR circumferential direction

ULR rotation direction

10 hollow body

11 braid

12 wire element

12′ inner wire element

12a first outer wire element

12b second outer wire element

12c third outer wire element

12d fourth outer wire element

12a′ first inner wire element

12b′ second inner wire element

12c′ third inner wire element

12d′ fourth inner wire element

12x edge-forming wire element

12x′ braid-forming wire element

13 first series

14a first terminal mesh

14b second terminal mesh

14c third terminal mesh

14d fourth terminal mesh

15 braid end

15a margin

16 terminating edge

16a first section

16b second section

16c front area

16d rear area

16e intermediate area

17b first location

17c second location

17c third location

18 terminal mesh

19 continuation

20 connecting element

21 wire bundle

22 distal end

23 “bobbin”

30 delivery system

31 catheter

32 actuation element/guide wire

33 decoupling mechanism

33a proximal end

33b release element

34 intermediate piece

35 holding means

35a first locking element

35b second locking element

36 inner mesh limit

37a first loop

37b second loop

37c second loop

38 open cell

39 apex

40 clot/concretion

50 blood vessel/hollow organ

1. A medical device for insertion into a hollow organ, comprising a hollow body that has a braid of wire elements with a series of terminal meshes which delimit an axial braid end, wherein the terminal meshes comprise outer wire elements, which form a terminating edge of the braid and merge into inner wire elements arranged within the braid, wherein a first section of the terminating edge and a second section of the terminating edge each have several outer wire elements which together form a peripheral margin of the terminating edge, which peripheral margin is adapted in such a way that the axial braid end of the hollow body can be refracted into a delivery system, wherein
the outer wire elements of the first section for forming the terminating edge are arranged directly one after another along the latter and each have a first axial component AK1, which extends in the longitudinal direction L of the hollow body, and
the outer wire elements of the second section for forming the terminating edge are arranged directly one after another along the latter and each have a second axial component AK2, which extends in the longitudinal direction L of the hollow body and is counter to the first axial component AK1, wherein the two axial components AK1, AK2 are in relation to the same rotation direction ULR of the margin.

2. The device as claimed in claim 1, wherein the outer wire elements of the first section have a first circumferential component UK1, and the outer wire elements of the second section have a second circumferential component UK2, wherein the first and second circumferential components UK1, UK2 extend in the same circumferential direction UR of the hollow body.

3. The device as claimed in claim 1, wherein the terminating edge is arranged obliquely with respect to the longitudinal direction L of the hollow body.

4. The device as claimed in claim 1, wherein at least one first outer wire element of a terminal mesh extends into the area of another terminal mesh, wherein the first outer wire element and at least one second outer wire element of the other terminal mesh overlap each other and are arranged together along the terminating edge.

5. The device as claimed in claim 4, wherein the at least one first outer wire element and the at least one second outer wire element are connected.

6. The device as claimed in claim 1, wherein the terminal meshes are formed by loops, which are staggered along the terminating edge and overlap each other, wherein the loops for forming the interconnected outer wire elements at the terminating edge are brought together at different locations that are arranged in succession along the terminating edge.

7. The device as claimed in claim 1, wherein the inner wire elements arranged in the braid are guided to the terminating edge or are branched off from the terminating edge.

8. The device as claimed in claim 7, wherein the inner wire elements at the transition to the outer wire elements change the direction of the circumferential component with respect to the circumferential direction and/or the direction of the axial component with respect to the longitudinal direction L of the hollow body.

9. The device as claimed in claim 7, wherein the inner wire elements and outer wire elements have the same braiding angle.

10. The device as claimed in claim 1, wherein the interconnected outer wire elements of the terminal meshes are connected by a form fit, in particular by twisting or intertwining, or by an integral bond, in particular by adhesive bonding, welding or soldering, or by mechanical connecting means, in particular by coils and/or sleeves and/or wires.

11. The device as claimed in claim 1, wherein the number of the outer wire elements of the first section and of the second section in each case increases toward a front area of the terminating edge in the longitudinal direction L of the hollow body and is at a maximum in the front area.

12. The device as claimed in claim 11, wherein the outer wire elements of the terminal meshes are brought together in a front terminal mesh.

13. The device as claimed in claim 12, wherein the outer wire elements form a single group, which is deflected in the area of the front terminal mesh.

14. The device as claimed in claim 12, wherein the outer wire elements form at least two groups, which are connected in the area of the front terminal mesh.

15. The device as claimed in claim 1, wherein the number of the outer wire elements of the first section and of the second section in each case decreases toward a front area of the terminating edge in the longitudinal direction L of the hollow body and is at a minimum in the front area.

16. The device as claimed in claim 15, wherein the outer wire elements of the first section and/or of the second section arranged remote from the front area of the terminating edge each form a wire bundle, which is arranged in the braid in order to strengthen the hollow body.

17. The device as claimed in claim 1, wherein an intermediate area is arranged between a front area and a rear area of the terminating edge, and the number of the outer wire elements, starting from the intermediate area, decreases toward the front area and toward the rear area of the terminating edge, wherein the number of the outer wire elements is at a maximum in the intermediate area.

18. The device as claimed in claim 1, wherein the inner wire elements form inner mesh limits of the terminal meshes of the first section and/or of the second section which, starting from the terminating edge, extend into the braid interior, wherein the terminal meshes are at least in part interconnected at the inner mesh limits.

19. The device as claimed in claim 18, wherein the outer wire elements of the terminal meshes do not overlap.

20. The device as claimed in claim 1, wherein the terminal meshes of the first section and/or of the second section in each case have at least two loops , which are arranged next to each other along the terminating edge, wherein at least a third loop overlaps the first loop and second loop.

21. The device as claimed in claim 1, wherein at least one outer wire element and/or an additional wire element forms a continuation which extends beyond the contour of the terminating edge.

22. A method for producing a medical device as claimed in claim 1, in which method a hollow body is braided from wire elements, and a series of terminal meshes arranged next to one another and delimiting an axial braid end is formed, wherein each terminal mesh comprises at least one outer wire element, and the outer wire elements of the terminal meshes together form a terminating edge of the braid end, wherein inner wire elements arranged in the braid, in order to form the outer wire elements, are deflected in such a way that the direction of winding and/or the direction of the axial component with respect to the longitudinal axis L of the inner wire elements is changed.
